(19) United States
(12) Patent Application Publication
(10) Pub. No.: US 2024/0390386 A1
(43) Pub. Date: Nov. 28, 2024

(54) CDK8/19 INHIBITORS FOR PREVENTING DRUG RESISTANCE

(71) Applicants: University of South Carolina, Columbia, SC (US); Senex Biotechnology, Inc., Columbia, SC (US)

(72) Inventors: Eugenia V. BROUDE, Lexington, SC (US); Igor B. RONINSON, Lexington, SC (US); Xiaokai DING, Cayce, SC (US); Thomas HILIMIRE, Columbia, SC (US); Zachary MACK, Columbia, SC (US)

(21) Appl. No.: 18/693,105

(22) PCT Filed: Sep. 16, 2022
(86) PCT No.: PCT/US2022/043852

§ 371 Date: Mar. 18, 2024

Related U.S. Application Data
(60) Provisional application No. 63/245,030, filed on Sep. 16, 2021.

(51) Int. Cl. A61K 31/551; A61K 31/436; A61K 31/4725; A61K 31/496; A61K 31/517; A61P 35/00

(52) U.S. Cl. CPC A61K 31/551; A61K 31/436; A61K 31/4725; A61K 31/496; A61K 31/517; A61P 35/00

(57) ABSTRACT

Disclosed herein are methods, compositions, and kits for treating a subject in need of a treatment for a cancer. The method comprises administering to the subject a CDK8/19 inhibitor and a mTOR inhibitor.

### CROSS-REFERENCE TO RELATED APPLICATIONS

This application claims priority to U. S. Provisional Application No. 63/245,030 filed on Sep. 16, 2021, the contents of which are incorporated by reference in its entirety.

### STATEMENT REGARDING FEDERALLY SPONSORED RESEARCH

This invention was made with government support under R44CA203184 awarded by National Institutes of Health. The government has certain rights in the invention.

### BACKGROUND OF THE INVENTION

Dysregulation of the PI3K/AKT/mTOR pathway is one of the most common genetic alterations in cancer, found in 38% of diverse solid tumors (Millis et al., 2016). This pathway is activated by receptor tyrosine kinases, which activate PI3K (phosphatidylinositol-3-kinase), followed by phosphorylation of AKT (a.k.a. protein kinase B) and mTOR (mammalian target of rapamycin) complex 1 (mTORC1). Oncogenic activation of the PI3K pathway can happen due to stimulation of upstream regulators (RTKs, cMET, RAF), activating mutations of PI3K catalytic subunit α (PIK3CA), or inactivation of negative regulators of PI3K, tumor suppressor PTEN and proline-rich inositol polyphosphatase (PIPP). Given the exceptional frequency of PI3K pathway activation in different cancers, major efforts have been devoted to the development of inhibitors of its key components: mTOR, PI3K and AKT (reviewed in (Kaur et al., 2021; Martorana et al., 2021; Mishra et al., 2021)). Several mTOR and PI3K inhibitors have been approved for oncological applications and others are in clinical trials, as are inhibitors of AKT. However, the benefits of PI3K/AKT/mTOR inhibitors have been generally limited, due to the rapid development of resistance to these agents (reviewed in (Formisano et al., 2020; Vitale et al., 2021)). As a result, there is a need for pharmacological approaches to prevent resistance to drugs targeting the PI3K/AKT/mTOR pathway.

### BRIEF SUMMARY OF THE INVENTION

Disclosed herein are methods and compositions for treating a subject in need of a treatment for a cancer, including but not limited to breast cancer. The method comprises administering to the subject a CDK8/19 inhibitor and a mTOR inhibitor.

Another aspect of the invention is a method for preventing resistance to an anticancer agent. The method comprises administering to a subject a CDK8/19 inhibitor and a mTOR inhibitor, wherein the subject is in need of a treatment for a cancer.

Another aspect of the invention provides for a pharmaceutical composition comprising a CDK8/19 inhibitor and a mTOR inhibitor.

Another aspect of the invention provides for kit comprising container containing a CDK8/19 inhibitor and a mTOR inhibitor.

These and other aspects of the invention will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Effects of SNX631 on MDA-MB-468 TNBC response to mTOR and AKT inhibitors in vitro. A. Effects of everolimus and SNX631, alone and in 1:2 mixture, on MDA-MB-468 cell growth (7-day SRB assay). B. Effects of capivasertib and SNX631, alone and in 1:2 mixture, on MDA-MB-468 cell growth (7-day SRB assay).

FIG. 2. Effects of SNX631 on MDA-MB-468 TNBC response to everolimus in vivo. A. Growth of MDA-MB-468 xenograft tumors receiving vehicle control, SNX631, everolimus, or SNX631+everolimus (mean+/−SEM). B. Growth of individual MDA-MB-468 xenograft tumors receiving everolimus or SNX631+everolimus (mean+/−SEM). C. KM plot of event-free survival in the same study; event defined as tumor volume reaching 500 mm³. D. Changes in mouse body weights of different groups (mean+/−SEM).

FIG. 3. RNA-Seq analysis of the effects of everolimus and SNX631 in MDA-MB-468 xenografts. A. Growth of MDA-MB-468 xenograft tumors in a short-term study of the effects of everolimus (mean+/−SEM). B. Effects of different treatments on the indicated genes in the short-term (ST, FIG. 2A) and long-term (LT, FIG. 1B) MDA-MB-368 xenograft studies. C. GSEA analysis of the effects of different treatments on hallmark genesets (affected with FDR<0.05) in tumor (human) cells in the same tumors.

FIG. 4. Effects of SNX631-6 on PEN061 TNBC PDX response to everolimus in vivo. A. Growth of PEN061 PDX tumors receiving vehicle control, SNX631-6, everolimus, or SNX631-6+everolimus (mean+/−SEM). B. Changes in mouse body weights of different groups (mean+/−SEM).

FIG. 5. Effects of SNX631-6 on PEN027 ER-positive breast cancer PDX response to everolimus in vivo. A. Growth of PEN027 PDX tumors receiving vehicle control, SNX631-6, everolimus, or SNX631-6+everolimus (mean+/−SEM). B. KM plot of event-free survival upon continuation of the same study; event defined as morbidity or tumor volume reaching 2000 mm³. C. Changes in mouse body weights of different groups (mean+/−SEM).

### DETAILED DESCRIPTION OF THE INVENTION

Disclosed herein are CDK8/19 inhibitors for preventing drug resistance to an anticancer agent, such as mTOR, AKT, or PI3K inhibitors. As demonstrated in the Examples, inhibitors of CDK8/19 are effective in preventing development of in vivo drug resistance to an anticancer agent targeting the PI3K/AKT/mTOR pathway.

Methods for treating subjects with a CDK8/19 inhibitor are provided. Suitably the methods, compositions, or kits for treating a subject comprise administering to the subject an effective amount of a CDK8/19 inhibitor (or CDK8/19i) and an effective amount of an anticancer agent or a pharmaceutical composition comprising the effective amount of a CDK8/19 inhibitor and the effective amount of an anticancer agent. The anticancer agent may include one or more agents that target the PI3K/AKT/mTOR pathway, such as a mTOR inhibitor, an AKT inhibitor, or a PI3K inhibitor. The CDK8/19i and anticancer agent may be administered together or at different times.

As used herein, a “subject” may be interchangeable with “patient” or “individual” and means an animal, which may be a human or non-human animal, in need of treatment. In particular embodiments, the subject is a human subject. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. In some embodiments, the subject is responsive to therapy with one or more of the compounds disclosed herein in combination with one or more additional therapeutic agents.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be treatment of the subject or preventing resistance to an anticancer agent, such as a mTOR inhibitor, an AKT inhibitor, or a PI3K inhibitor.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A “subject in need of treatment” may include a subject having a disease, disorder, or condition that may be characterized as a cancer. In some embodiments, the cancer is a cancer that can be treated with an anticancer agent, such as a mTOR inhibitor, an AKT inhibitor, or a PI3K inhibitor. Exemplary cancers include breast cancers, such as triple-negative breast cancer or estrogen receptor positive (ER+) breast cancer or HER2-positive breast cancers. Resistance to both conventional and targeted drugs involves metastable transcriptional changes that allow tumor cells to adapt and survive drug exposure. Transcriptional reprogramming is a key feature of tumor cell plasticity, which allows the cells to grow under adversarial conditions (treatment resistance) and in a heterologous environment (metastasis). This non-genetic resistance of tumor cell populations provides the background for subsequent selection of stable genetic variants that yield higher levels of resistance. Enhancement to anticancer therapy may be realized by co-administering a CDK8/19 inhibitor with one or more anticancer agents.

In some embodiments, the subject has a cancer or solid tumor possessing a genetic alteration associated with PI3K/AKT/mTOR dysregulation. Dysregulation of the PI3K/AKT/mTOR pathway is one of the most common genetic alterations in cancer and found in 38% of diverse solid tumors. Given the frequency of activation of this pathway in different cancers, anticancer agents that target this pathway have been sought. However, the benefits of these anticancer agents are limited, due to rapid development of resistance to these anticancer agents. The present technology advantageously prevents or mitigates resistance to an anticancer agent, such as a mTOR inhibitor, an AKT inhibitor, or a PI3K inhibitor, thereby extending the efficacy of the anticancer agent.

The anticancer agent is co-administered with a CDK8/19 inhibitor. CDK8 (ubiquitously expressed) and CDK19 (expressed in some cell types) (Tsutsui et al., 2011) are two isoforms of Mediator kinase, the enzymatic component of the CDK module that binds to the transcriptional Mediator protein complex. In addition to CDK8 or CDK19, the CDK module includes Cyclin C, MED12 and MED13 (Fant and Taatjes, 2019; Philip et al., 2018). Unlike better-known CDKs (such as CDK1, CDK2 or CDK4/6), CDK8/19 regulate transcription but not cell cycle progression. In contrast to other transcriptional CDKs, such as CDK7 or CDK9, Mediator kinase is not a part of the overall transcription machinery (Fant and Taatjes, 2019) but acts as a cofactor or modifier of several cancer-relevant transcription factors, including β-catenin/TCF/LEF (Firestein et al., 2008), SMADs (Alarcon et al.; Serrao et al.), Notch (Fryer et al., 2004), STATs (Bancerek et al., 2013), HIFIA (Galbraith et al., 2013), ER (McDermott et al., 2017), NFκB (Chen et al., 2017) and MYC (Adler et al., 2012; Andrysik et al., 2021; EV et al., 2015; Fukasawa et al., 2021). CDK8/19 Mediator kinase directly phosphorylates some transcription factors (SMADs, STATs, Notch) and in other cases acts through C-terminal phosphorylation of RNA polymerase II (Pol II), enabling the elongation of transcription. Importantly, CDK8/19 affect Pol II phosphorylation not globally but only in the specific context of newly induced genes (Chen et al., 2017; Donner et al., 2010; Galbraith et al., 2013), impacting primarily de novo-induced but not basal transcription (Chen et al., 2017; McDermott et al., 2017). Based on this unique activity, CDK8/19 were identified as regulators of transcriptional reprogramming (Chen et al., 2017; Fant and Taatjes, 2019; Steinparzer et al., 2019).

Administration of a CDK8/19i may prevent the emergence of resistance to an anticancer agent. As used herein, “prevent resistance to an anticancer agent” means that the duration of the anticancer agent effectiveness is extended by a statistically significant amount or at least 25%. In some embodiments, the duration of effectiveness may be extended by 50%, 100%, 150%, 200%, or more. As demonstrated in the Examples, tumor xenografts developed resistance to the anticancer agent acting on mTOR after about 50 days of treatment. Co-administration of the anticancer agent with the CDK8/19i prevented the development of in vivo resistance to the anticancer agent, extending the duration of effectiveness for the full length of the study. Thus, the duration of effectiveness was extended by at least about 200%, i.e., to day 150 when the study was ended.

The Combination Index (CI) for the CDK8/19 inhibitor and the anticancer agent in a tumor cell line may be less than 1, indicating synergy. CI provides a quantitative measure of the extent of drug interaction at a given effect level. That is, the combination concentrations of drug A and drug B to produce an effect x, C_{A,x} and C_{B,x}, are normalized by their corresponding concentrations that produces the same effect as a single agent, IC_{x,A} and IC_{x,B}, respectively. The sum of C_{A,x}/IC_{x,A} and C_{B,x}/IC_{x,B} is defined as the combination index at effect x. A CI less than 1.0 indicates synergy. In some embodiments, the CI for the CDK8/19i and the anticancer agent is less than 1.0, for example from about 0.1 to 1.0, 0.1 to 0.9, 0.1 to 0.8, 0.1 to 0.7, 0.1 to 0.6, or 0.1 to 0.5 for an effective dose (ED), e.g., an ED₂₅, ED₅₀, ED₇₅, ED₉₀, or ED₉₅. CI in a tumor cell line may be determined by measuring the relative cell number by sulforhodamine B (SRB) assay at 570 nm wavelength light absorbance or other by methods known in the art.

Determining the CI for the CDK8/19 inhibitor and the anticancer agent at an effective dose in a tumor cell line may be used to identify combinations of the CDK8/19 inhibitor and anticancer agent that have an improved or synergistic effect against the tumor cell line. In the Examples, the relative MDA-MB-468 cell number was determined, but t other cell lines for determining the CI may be utilized. The tumor cell line may be responsive to the anticancer agent and determining the CI may be used to identify tumors or cancers that may be treated by the combination of the anticancer agent and a CDK8/19 inhibitor. The response of the tumor cell line to the anticancer agent may be to kill tumor cells or inhibit the growth or proliferation of the tumor cells. By way of example, the assayed tumor cell line may be responsive to mTOR inhibitors and determining CI may be used to identify tumors or cancers that may be treated by the combination of the mTOR and a CDK8/19 inhibitor. Suitably, the tumor cell line is one that is responsive to one or more mTOR inhibitors, AKT inhibitors, and/or PI3K inhibitors or has a genetic alteration associated with PI3K/AKT/mTOR dysregulation.

### CDK8/19 Inhibitors

The Examples presented herein demonstrate the utility of the inhibitors of transcription-regulating kinases CDK8 and CDK19 (reviewed in (Fant and Taatjes, 2018; Menzl et al., 2019; Philip et al., 2018)) for preventing resistance to anticancer agents. 3-amino-4-(4-(4 (dimethylcarbamoyl)phenyl)-1,4-diazepan-1-yl)thieno[2,3-b]pyridine-2-carboxamide (SNX631) and 3-amino-4-(4-(4-(bis(methyl-d3) carbamoyl)phenyl)-1,4-diazepan-1-yl)thieno[2,3-b]pyridine-2-carboxamide (SNX631-D6) were used in the Examples but other inhibitors of CDK8 and CDK19 may also be used in the presently disclosed technology, such as 4-[2-[6-(4-methylpiperazine-1-carbonyl) naphthalen-2-yl]ethylamino]quinazoline-6-carbonitrile (Senexin B; 1449228-40-3), 4-[2-[6-(4-methylpiperazine-1-carbonyl) naphthalen-2-yl]ethylamino]quinoline-6-carbonitrile (Senexin C), (1S,2R,5S,6R,12R, 13R, 14S, 16R)-14-(dimethylamino)-5-isoquinolin-7-yl-6-methyl-19-oxapentacyclo[14.2.1.01,9.02,6.011,16]nonadeca-8,10-diene-12,13-diol (cortistatin A; CAS 882976-95-6) (Pelish et al., 2015), 2-[4-(4-isoquinolin-4-ylphenyl) pyrazol-1-yl]-N,N-dimethylacetamide (BI-1347) (Hofmann et al., 2020),.04,12]dodeca-2,4,6,8 (12)-tetraene (SEL120-34A; CAS 1609522-33-9) (Rzymski et al., 2017). Additional exemplary CDK8/19 inhibitors that may be used in the presently disclosed technology are disclosed in U. S. Pat. Nos. 8,598,344, 9,321,737, 9,409,873; US 2020/0062728, WO 2019/168446; WO 2020/160537; WO 2020/237014, (Ma et al., 2020; Philip et al., 2018; Xi et al., 2019); (Al-Sanea, 2020; Grandjean et al., 2020; Hofmann et al., 2020; Li et al., 2020; Martínez-González et al., 2020; Solum et al., 2020; Yu et al., 2021); and Wu et al., 2021, the contents of each is incorporated by reference in their entirety. In particular embodiments, the methods, pharmaceutical compositions, and kits use SNX631, SNX631-6, Senexin B, Senexin C, BI-1347, cortistatin A, SEL120-34A, or any combination thereof.

In some embodiments, the CDK8/19 inhibitor selectively inhibits CDK8 and CDK19. As used herein, an inhibitor that “selectively inhibits CDK8 and CDK19” is a compound that inhibits CDK8 and CDK19 without inhibiting the majority of other kinases. Selective inhibition can be determined by kinome profiling using an active site-directed competition binding assay to quantitatively measure interactions between the compound and a plurality of human kinases and disease relevant mutant variants. In some embodiments, the inhibitor that selectively inhibits CDK8 and CDK19 has an S-score of S(35)<0.1 or S(10)<0.01 at an effective amount of the CDK8 and CDK19 inhibitor, where S(#)=(number of non-mutant kinases with % Ctrl (or POC) <#)/(number of non-mutant kinases tested). In some embodiments, the inhibitor that selectively inhibits CDK8 and CDK19 has an S-score of S(35)<0.08, 0.06, 0.04, or 0.02. In some embodiments, the inhibitor that selectively inhibits CDK8 and CDK19 has an S-score of S(10)<0.080, 0.006, or 0.004. For example, SNX631 has a S(35) and S(10) against a panel of 468 kinases of less than 0.02 and 0.004, respectively, at 2000 nM (WO 2020/237014).

mTOR Inhibitors

In some embodiments, the anticancer agent is an mTOR inhibitor. mTOR inhibitors approved for cancer therapy include rapamycin analogs (rapalogs), such as everolimus (CAS 159351-69-6), approved for estrogen receptor (ER)-positive breast cancers (in combination with an aromatase inhibitor) and for pancreatic neuroendocrine tumors (NET) as a single agent, temsirolimus (CAS 162635-04-3) approved for renal cell carcinoma, sirolimus (CAS 53123-88-9), ridaforolimus (CAS 572924-54-0), umirolimus (CAS 851536-75-9), deforolimus (CAS 572924-54-0), zotarolimus (CAS 221877-54-9), and nanoparticle albumin-bound rapamycin. Rapalogs act as FKBP12-dependent allosteric inhibitors that inhibit only mTORC1 but not mTORC2 complex. Rapalog resistance can arise due to incomplete allosteric inhibition of mTORC1, activation of mTORC2, stimulation of PI3K/AKT signaling, and occasionally mutations in the mTOR FRB domain (Formisano et al., 2020). Additional mTOR inhibitors act directly on mTOR kinases and inhibit both mTORC1 and mTORC2 such as PP242, PP30, and others as described in Zhou and Huang (2012), the contents of which is incorporated by reference in their entirety.

Additional exemplary mTOR inhibitors to the rapalogs described above include, without limitation: PP242 (CAS 1092351-67-1), PP30 (CAS 1092788-09-4), PI103 (CAS 371935-74-9), NVPBEZ235 (CAS 915019-65-7), XL765 (CAS 934493-76-2), WJD008 (CAS 1309087-83-9), SF-1126 (CAS 936487-67-1), Torin1 (CAS 1222998-36-8), Ku-0063794 (CAS 938440-64-3), WYE-354 (CAS 1062169-56-5), WAY-600 (CAS 1062159-35-6), WYE-687 (CAS 1062161-90-3), AZD8055 (CAS 1009298-09-2), OSI-027 (CAS 936890-98-1), INK128 (CAS 1224844-38-5), GDC-0349 (CAS 1207360-89-1), GNE-555, PF-05139962 (CAS 1393712-18-9), AZD2014 (CAS 1009298-59-2), Torin 2 (CAS 1223001-51-1), MLN0128 (CAS 1224844-38-5), OXA-01 (CAS 936889-68-8), XL388 (CAS 1251156-08-7), CC214-1 (CAS 1021920-32-0), CC-223 (CAS 1228013-30-6), CC-115 (CAS 1228013-15-7), DHM25 (CAS 1685280-21-0), gedatolisib (CAS 1197160-78-3) as well as other described in Mao, et al., 2022. and others described in (Mao et al., 2022). Some of these inhibitors, however, are neutralized by the activation of PI3K/AKT, downregulation of eIF4E-binding proteins, or by transcriptional changes, such as the activation of FOXO family transcription factors (Formisano et al., 2020).

In particular embodiments, the methods, pharmaceutical compositions, and kits may use everolimus, temsirolimus, sirolimus, ridaforolimus, umirolimus, gedatolisib, or any combination thereof.

### PI3K Inhibitors

In some embodiments, the anticancer agent comprises a PI3K inhibitor. Among PI3K inhibitors, alpelisib (BYL719; CAS 1217486-61-7), a selective inhibitor of PI3Kα, has been approved for the treatment of ER+/HER2− metastatic breast cancer patients harboring a PIK3CA mutation, copanlisib (CAS 1217486-61-7) for follicular lymphomas (FL), duvelisib (CAS 1201438-56-3) for chronic lymphocytic leukemia/small lymphocytic leukemia (CLL/SLL) and idelalisib (CAS 870281-82-6) for CLL/SLL and FL (Vitale et al., 2021). As with mTOR inhibitors, resistance to PI3K inhibitors inevitably emerges, through PI3K/AKT/mTOR pathway reactivation by several mechanisms, MYC pathway activation, and activation of compensatory pro-survival mechanisms, including MAPK/MEK upregulation, activation of STAT5 or STAT3, and upregulation of PIM1, AXL or TCF7 transcription factor, a positive regulator of WNT/β-catenin signaling (Vitale et al., 2021).

### AKT Inhibitors

In some embodiments, the anticancer agent comprises an AKT inhibitor. The serine/threonine kinase AKT is a key component of the PI3K/AKT/mTOR signaling pathway as it exerts a pivotal role in cell growth, proliferation, survival, and metabolism. Deregulation of this pathway is a common event in breast cancer including hormone receptor-positive (HR+) disease, HER2-amplified, and triple negative tumors. Exemplary AKT inhibitors include, without limitation, capivasertib (CAS 870281-82-6) and ipatasertib (CAS 1001264-89-6).

### Combined Inhibitors

Combining inhibitors of different PI3K/AKT/mTOR pathway components yields a strong synergistic activity, as demonstrated for inhibitors of mTOR and AKT (Woo et al., 2017) and mTOR and PI3K (Elkabets et al., 2013). Towards this end, dual PI3K/mTOR inhibitors have been developed, among which gedatolisib is currently in clinical trials (Colombo et al., 2021). The efficacy of dual inhibitors, however, is also limited by the onset of resistance, through activation of Foxo transcription, RAF/MEK/ERK, JAK2/STAT5 and ER-dependent transcription in ER+breast cancer cells (Formisano et al., 2020). Hence, multiple regulatory mechanisms, most of which center on transcriptional reprogramming, combine to generate resistance to different PI3K/AKT/mTOR inhibitors. An approach to suppressing these transcriptional resistance mechanisms could revitalize the utility of such inhibitors, with a potentially transformative impact for a very large fraction of all cancer patients.

### Pharmaceutical Compositions

The CDK8/19 inhibitors and anticancer agents disclosed herein may be formulated as a single pharmaceutical composition or separate pharmaceutical compositions that include: an effective amount of one or more compounds and one or more pharmaceutically acceptable carriers, excipients, or diluents. The pharmaceutical composition may include the CDK8/19 inhibitors and anticancer agents in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The CDK8/19 inhibitors and anticancer agents utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The CDK8/19 inhibitors and anticancer agents utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, nano- or microparticles, and starch-gelatin paste.

The CDK8/19 inhibitors and anticancer agents utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The CDK8/19 inhibitors and anticancer agents utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The CDK8/19 inhibitors and anticancer agents utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The CDK8/19 inhibitors and anticancer agents employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the compounds or salts, esters, and/or amides, thereof. Solvate forms may include ethanol solvates, hydrates, and the like.

### Kits

Also disclosed herein are kits comprising a container containing a CDK8/19 inhibitor and an anticancer agent. The anticancer agent may include one or more agents that target the PI3K/AKT/mTOR pathway, such as a mTOR inhibitor. The kit may contain a single pharmaceutical composition or separate pharmaceutical compositions as described herein. Suitably, the kit may contain an effective amount of one or more compounds and one or more pharmaceutically acceptable carriers, excipients, or diluents. In some embodiments, the kit comprises a pharmaceutical composition comprising a CDK8/19 inhibitor, a mTOR inhibitor, and a pharmaceutically acceptable carrier, excipient, or diluent. In other embodiments, the kit comprises a first pharmaceutical composition comprising a CDK8/19 inhibitor and a pharmaceutically acceptable carrier, excipient, or diluent and a second pharmaceutical composition comprising a mTOR inhibitor and a pharmaceutically acceptable carrier.

The kit may further comprise instructions for administering the CDK8/19 inhibitor and the anticancer agent to the subject. The instructions may provide the dosing schedule for administering the CDK8/19 inhibitor and the anticancer agent. Suitably, the CDK8/19 inhibitor and the anticancer agent may be administered with the same, e.g., each administered once or twice a day, or different dosing schedule, e.g., one of the compounds administered once a day and the other compound administered twice a day.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Example 1. CDK8/19 Inhibition Prevents the Development of In Vivo Resistance to an mTOR Inhibitor in a Cell Line-Based Xenograft Model of Triple-Negative Breast Cancer (TNBC)

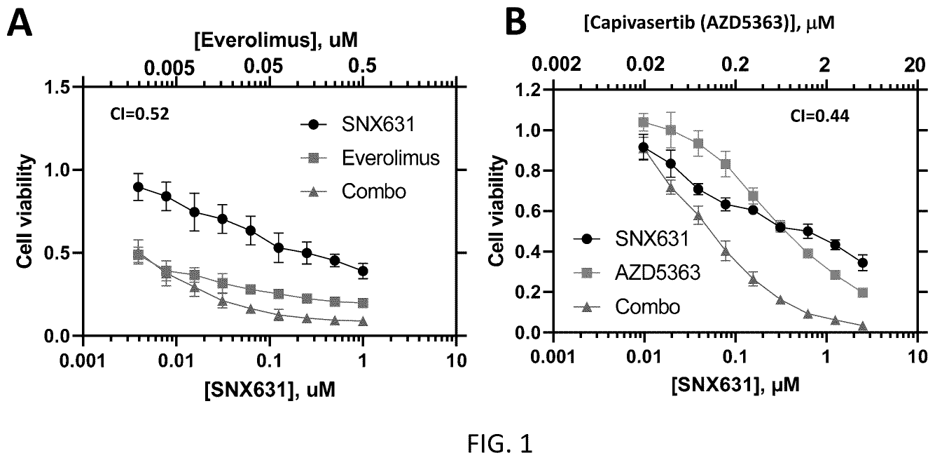

The effect of CDK8/19i and an mTOR inhibitor on the growth of triple-negative breast cancer (TNBC) cells was investigated. MDA-MB-468 TNBC cell line is PTEN-deficient (Meric-Bernstam et al., 2012) and therefore sensitive to everolimus. CDK8/19 inhibitor SNX631 (a.k.a. 15u) (Ding et al., 2022) (PCT/US2020/016394 and PCT/US2020/033937) was tested for synergy with mTOR inhibitor everolimus in these cells in vitro. In the experiment in FIG. 1A, 1,000 MDA-MB-468 cells were plated per well in 96-well plates. Cells were treated with serial dilutions of everolimus, SNX631 and their 1:2 mixture (Combo), for 7 days. Relative cell number was measured by sulforhodamine B (SRB) assay, at 570 nm wavelength light absorbance. This analysis provided evidence of synergy, based on CompuSyn (Chou, 2010) determination of Combination Index (CI) at ED75 of 0.52 (CI<1 is considered synergistic).

The same analysis was used to determine the interaction between SNX631 and AKT inhibitor capivasertib (a.k.a. AZD5363) in MDA-MB-468 cells (FIG. 1B). This analysis also provided evidence of synergy between the two inhibitors, with CI of 0.44.

We then investigated combinatorial effects of everolimus and SNX631 on in vivo growth of MDA-MB-468 xenografts. Female NSG mice were injected with MDA-MB-468 cells in the fat pad. When tumors became palpable, mice were randomized into 4 groups (n=12) receiving control vehicle (70% PEG400/30% Propylene Glycol), SNX631 in medicated food (350 ppm), mTOR inhibitor everolimus (2 mg/kg by daily gavage in the control vehicle), or a combination of SNX631 and everolimus. Tumor volumes were measured with calipers twice a week and mouse body weights were measured.

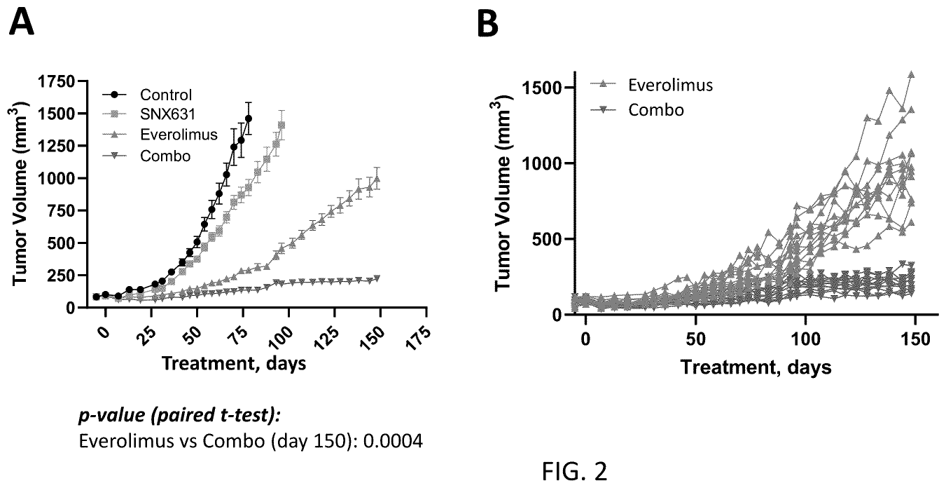

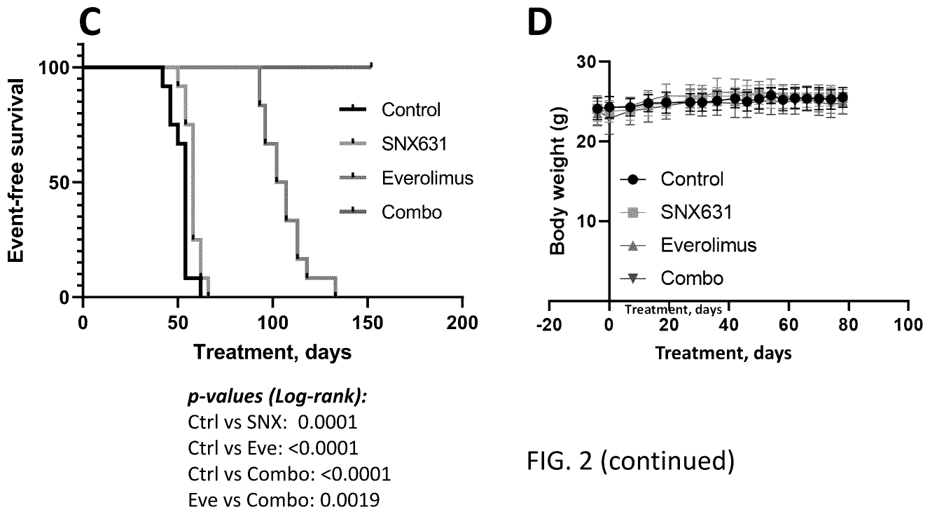

SNX631 alone had a modest effect on tumor growth, while everolimus alone fully suppressed xenograft growth until ˜50 days of treatment, but later on all the tumors developed everolimus resistance (FIG. 2A-C), resembling the development of resistance in clinical trials of mTOR inhibitors in TNBC (Costa et al., 2018). The addition of SNX631, however, completely prevented the development of in vivo resistance to everolimus over the 150 days of the study, as indicated both by average and individual tumor volumes (FIG. 2A,B) and by Kaplan-Meier (KM) analysis of event-free survival, where the event is defined by the tumor volume reaching 500 mm³ (FIG. 2C). The CDK8/19 and mTOR inhibitors, as well as their combination, were very well tolerated, based on mouse body weights (FIG. 2D).

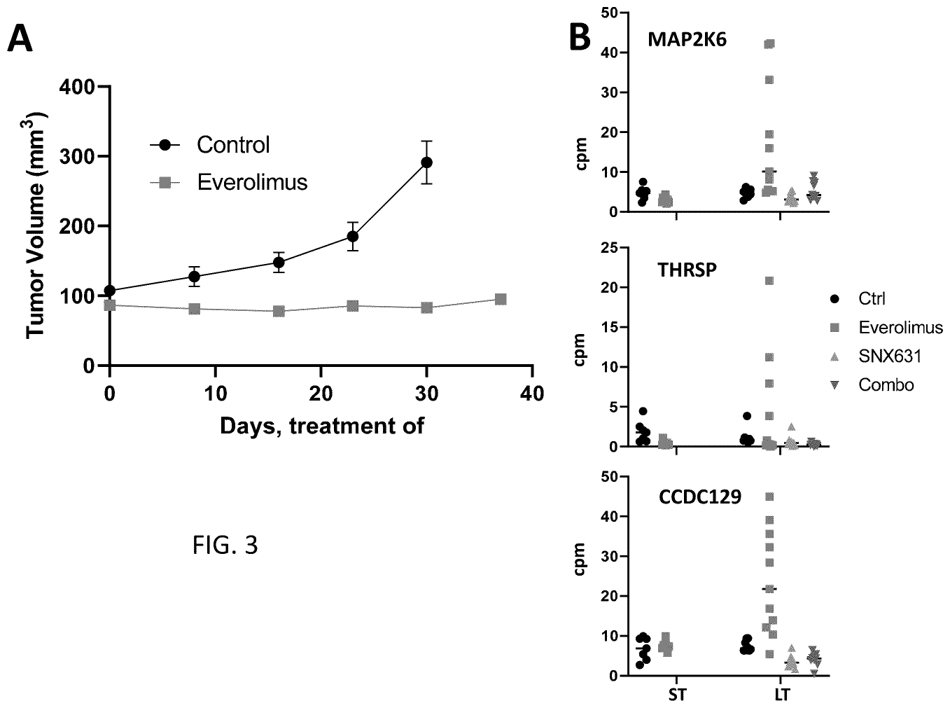

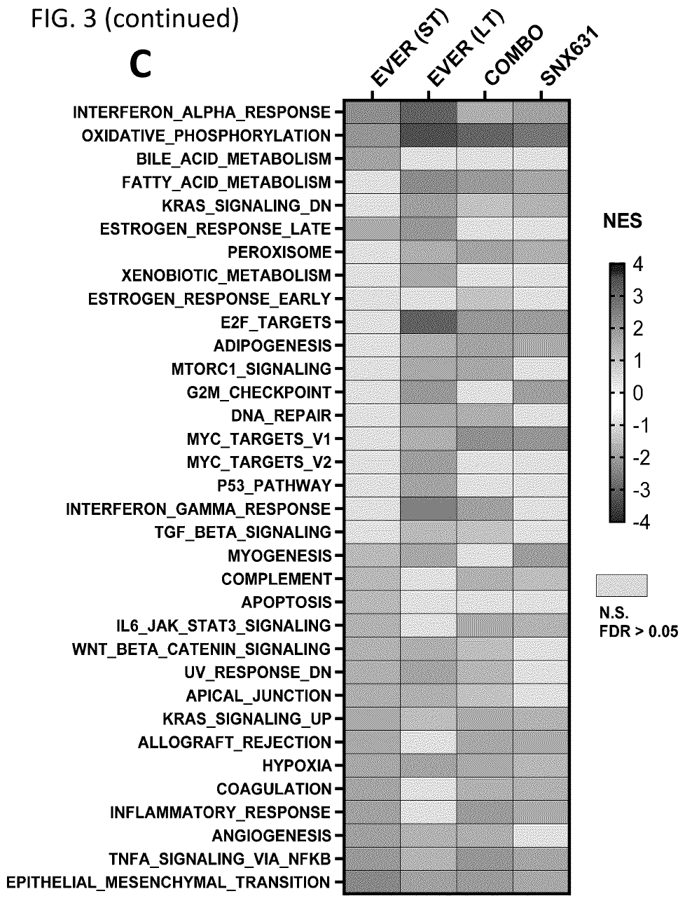

To elucidate the mechanism of the prevention of in vivo resistance to everolimus, RNA-Seq analysis was carried out on xenograft tumors from different treatment groups in the long-term (LT) study in FIG. 2A-D and in a subsequent short-term (ST) study, where control and everolimus-treated tumors (n=7-8) were collected after 30 and 37 days of treatment, respectively, when the everolimus-treated tumors were still growth-inhibited (FIG. 3A). RNA-Seq was carried out as described (Ding et al., 2022). Results of RNA-Seq analysis are shown in FIG. 3B,C, illustrating that prevention of everolimus resistance was associated with the inhibition of transcriptional reprogramming by the CDK8/19 inhibitor. Thus, some of the genes that were upregulated in individual everolimus-selected tumors were not upregulated before the tumors developed resistance. Examples of such genes (FIG. 3B) include MAP2K6 (MKK6), a member of MAPK family implicated in resistance to mTOR inhibitors (Li et al., 2012), THRSP associated with poor outcome in invasive breast cancers (Wells et al., 2006) and promoting mammary carcinogenesis (Wellberg et al., 2014) and CCDC129. Induction of such potentially resistance-associated genes was prevented when everolimus was combined with SNX631. GSEA analysis of 50 hallmark pathways (FIG. 3C) showed that combined everolimus-resistant tumors show many altered pathways but adding SNX631 to the treatment regimen reverses the effects of everolimus selection on several key pathways (such as INFα, KRAS, p53 and IFNγ).

Given the drastic effect of the CDK8/19 inhibitor in preventing the development of in vivo resistance to everolimus in MDA-MB-468 xenografts, via the regulation of transcription, CDK8/19 inhibitors will prevent resistance not only to mTOR but also to PI3K and AKT inhibitors (such as are listed in (Kaur et al., 2021; Martorana et al., 2021; Mishra et al., 2021)), as well as to mTOR, AKT and PI3K inhibitor combinations, drastically increasing the benefits of PI3K/AKT/mTOR-targeting drugs that have not yet fulfilled their potential. With the very high frequency of PI3K/AKT/mTOR pathway aberrations across different solid tumor types, preventing the resistance to the inhibitors of this pathway by blocking CDK8/19-mediated transcriptional reprogramming can have a major impact on extending patient survival in different types of cancer.

### Example 2. CDK8/19 Inhibition Suppresses the Development of In Vivo Resistance to an mTOR Inhibitor in a Patient-Derived Xenograft (PDX) TNBC Model

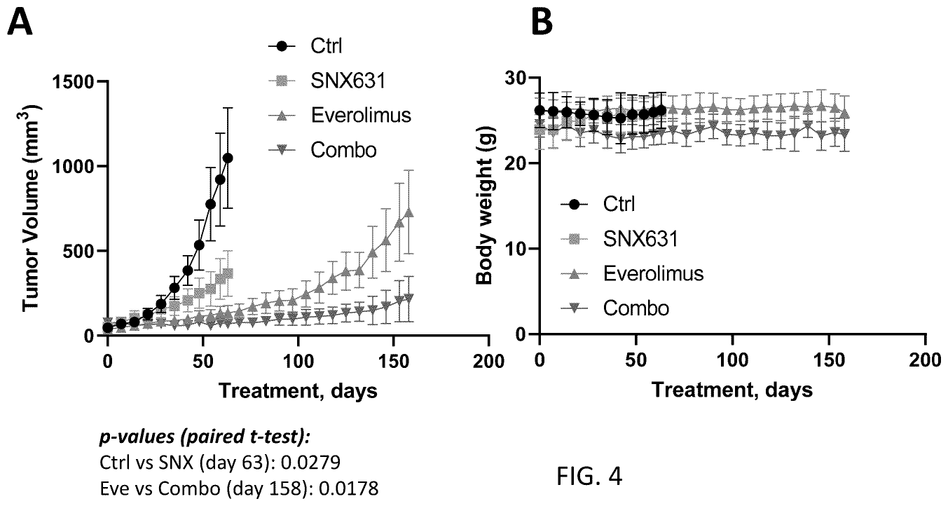

We then determined if the effects of CDK8/19 inhibition on the development of everolimus resistance observed in a xenograft model derived from a clonal cell line would also be observed in a clinically relevant patient-derived xenograft (PDX) model that retains much of the heterogeneity of the original patient's tumor, and if the effects of SNX631 could be reproduced with another CDK8/19 inhibitor. PEN061 PDX TNBC model and expanded by transplantation in female NSG mice. After expansion, tumors were excised, cut into small pieces, and re-implanted subcutaneously into the right flank of female NSG mice. Once tumors became palpable, mice were randomly allocated to four groups (n=8-11) and treated with control vehicle (the same as in Example 1), CDK8/19 inhibitor SNX631-6 (a.k.a. 15u-D6) (PCT/US2020/016394 and PCT/US2020/033937) in medicated food (350 ppm), everolimus (3 mg/kg by daily gavage), or a combination of SNX631-6 and everolimus. Tumor growth and body weights were monitored as in Example 1. As shown in FIG. 4A, after 63 days of treatment, SNX631-6 alone strongly inhibited tumor growth but the tumor-suppressive effect of everolimus alone or SNX631-6+everolimus combination was even stronger. Mice of the control and SNX631-6 groups were sacrificed on day 63 but the everolimus and combination arms were continued up to 158 days, by which time many of the tumors treated with everolimus alone developed resistance but the resistance was much less prominent in the combination-treated group, resulting in a highly significant difference between the tumor volumes in these two arms (FIG. 4A). Average mouse body weights in each group are plotted in FIG. 4B, indicating lack of significant toxicity of the treatments with individual drugs or their combination.

### Example 3. CDK8/19 Inhibition Suppresses the Development of In Vivo Resistance to an mTOR Inhibitor in a PDX Model of Estrogen Receptor (ER)-Positive Breast Cancer

In the next study, we determined if CDK8/19 inhibition suppresses the development of everolimus resistance in a PDX model of ER-positive breast cancer, the type of cancer where everolimus has been approved for clinical use. PEN027 PDX TNBC model was expanded by transplantation in female NSG mice. This PDX model was exceptionally aggressive and its growth in female NSG mice was too rapid and heterogeneous to monitor treatment effects. To improve the sensitivity of this model for therapeutic studies, we transplanted the tumors to male nude mice (modelling clinical breast cancers in men, which are mostly ER-positive (Khan et al., 2015)), where these PDX tumors still grew fast but not as fast as in females.

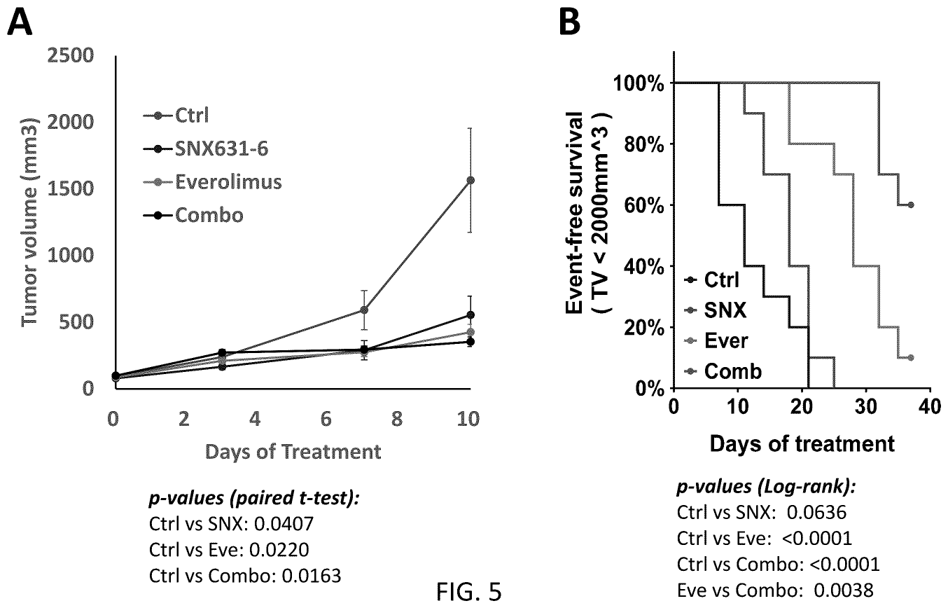

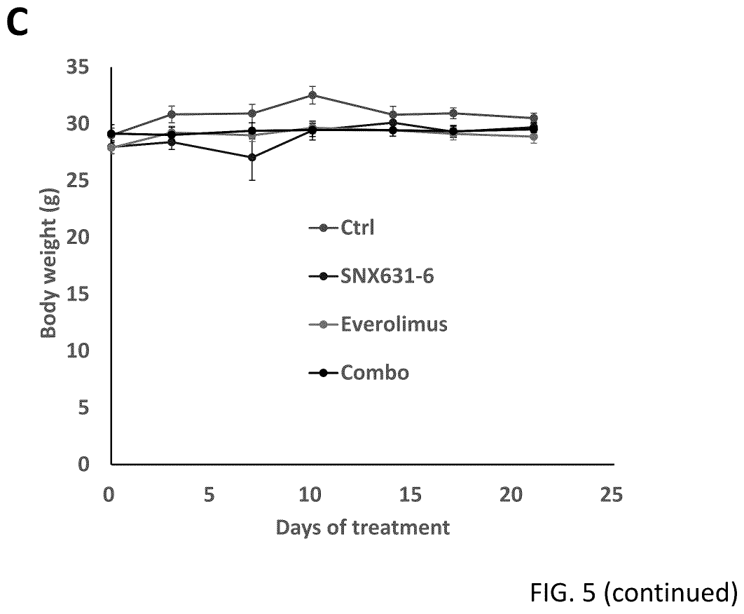

Once PEN027 tumors in male nude mice became palpable, mice were randomly allocated to four groups (n=10) and then treated with control vehicle, everolimus (2 mg/kg daily gavage), SNX631-6 (500 ppm medicated food) and everolimus+SNX631-6 combination. FIG. 5A shows the effects of different treatments on tumor growth over the first 10 days of treatment, at which point some of the control tumors exceeded 2,000 mm³ volume requiring euthanasia. SNX631-6, everolimus and their combination all significantly inhibited tumor volume at this point (FIG. 5A). The study was subsequently continued as a survival study. FIG. 5B shows the KM plot of event-free survival (event defined as tumors reaching 2,000 mm³ or mice requiring euthanasia) after 38 days of treatment. SNX631-6 treatment had only a moderate effect on survival, whereas everolimus had a strong effect. Everolimus-treated mice eventually succumbed but the effect of everolimus on survival was strongly improved by the combination with SNX631-6 (FIG. 5B). FIG. 5C plots the time course of body weight changes of the surviving mice, indicating lack of significant toxicity of any treatment.

In summary, Examples 1-3 demonstrate that combining a mTOR inhibitor with different inhibitors of CDK8/19 drastically improves the long-term outcome of mTOR inhibitor treatment in a cell-line based and patient-derived xenograft models of TNBC and ER-positive breast cancer, preventing or delaying the development of resistance to the mTOR inhibitor. Furthermore, the combination of CDK8/19 and mTOR inhibitors showed no apparent toxicity over very long periods of treatment (up to 158 days), which is highly unusual for cancer drug combinations. These results indicate that combining CDK8/19 and mTOR inhibitors can be uniquely beneficial for the treatment of different types of cancer, especially breast cancer.

### REFERENCES

Adler, A. S., McCleland, M. L., Truong, T., Lau, S., Modrusan, Z., Soukup, T. M., Roose-Girma, M., Blackwood, E. M., and Firestein, R. (2012). CDK8 maintains tumor dedifferentiation and embryonic stem cell pluripotency. Cancer Res 72, 2129-2139. 0008-5472.CAN-11-3886 [pii]; 10.1158/0008-5472.CAN-11-3886 [doi].
Al-Sanea, M. M. (2020). Synthesis and biological evaluation of small molecule modulators of CDK8/Cyclin C complex with phenylaminoquinoline scaffold. PeerJ 8, e8649. 10.7717/peerj.8649.
Alarcon, C., Zaromytidou, A. I., Xi, Q., Gao, S., Yu, J., Fujisawa, S., Barlas, A., Miller, A. N., Manova-Todorova, K., Macias, M. J., et al. (2009). Nuclear CDKs drive Smad transcriptional activation and turnover in BMP and TGF-beta pathways. Cell 139, 757-769.
Andrysik, Z., Bender, H., Galbraith, M. D., and Espinosa, J. M. (2021). Multi-omics analysis reveals contextual tumor suppressive and oncogenic gene modules within the acute hypoxic response. Nat Commun 12, 1375. 10.1038/s41467-021-21687-2.
Balkwill, F. (2004). Cancer and the chemokine network. Nat Rev Cancer 4, 540-550. 10.1038/nrc1388.
Bancerek, J., Poss, Z. C., Steinparzer, I., Sedlyarov, V., Pfaffenwimmer, T., Mikulic, I., Dolken, L., Strobl, B., Muller, M., Taatjes, D. J., and Kovarik, P. (2013). CDK8 kinase phosphorylates transcription factor STAT1 to selectively regulate the interferon response. Immunity 38, 250-262. S1074-7613 (13) 00009-5 [pii]; 10.1016/j.immuni.2012.10.017 [doi].
Broude, E. V, Gyorffy, B., Chumanevich, A. P., Chen, M., McDermott, M. S., Shtutman, M., Catroppo, J. F., and I. B., R. (2015). Expression of CDK8 and CDK8-interacting genes as potential biomarkers in breast cancer. Curr. Cancer Drug Targets 15 (8): 739-49. doi: 10.2174/156800961508151001105814.
Chen, B., Wen, P., Hu, G., Gao, Y., Qi, X., Zhu, K., Chen, S., Wu, L., Xu, A., and Zhao, G. (2020). Antagonizing CDK8 Sensitizes Colorectal Cancer to Radiation Through Potentiating the Transcription of e2f1 Target Gene apaf1. Front Cell Dev Biol 8, 408. 10.3389/fcell.2020.00408.
Chen, M., Liang, J., Ji, H., Yang, Z., Altilia, S., Hu, B., Schronce, A., McDermott, M. S. J., Schools, G. P., Lim, C. U., et al. (2017). CDK8/19 Mediator kinases potentiate induction of transcription by NFkappaB. Proc Natl Acad Sci USA 114, 10208-10213. 10.1073/pnas. 1710467114.
Chou, T. C. (2010). Drug combination studies and their synergy quantification using the Chou-Talalay method. Cancer Res 70, 440-446. 0008-5472.CAN-09-1947 [pii]; 10.1158/0008-5472.CAN-09-1947 [doi].
Colombo, I., Genta, S., Martorana, F., Guidi, M., Frattini, M., Samartzis, E. P., Brandt, S., Gaggetta, S., Moser, L., Pascale, M., et al. (2021). Phase I Dose-Escalation Study of the Dual PI3K-mTORC1/2 Inhibitor Gedatolisib in Combination with Paclitaxel and Carboplatin in Patients with Advanced Solid Tumors. Clin Cancer Res. 10.1158/1078-0432.Ccr-21-1402.
Costa, R. L. B., Han, H. S., and Gradishar, W. J. (2018). Targeting the PI3K/AKT/mTOR pathway in triple-negative breast cancer: a review. Breast Cancer Res Treat 169, 397-406. 10.1007/s10549-018-4697-y.
Ding, X., Sharko, A. C., McDermott, M. S. J., Schools, G. P., Chumanevich, A., Ji, H., Li, J., Zhang, L., Mack, Z. T., Sikirzhytski, V., et al. (2022). Inhibition of CDK8/19 Mediator kinase potentiates HER2-targeting drugs and bypasses resistance to these agents in vitro and in vivo. Proc Natl Acad Sci USA 119, e2201073119. 10.1073/pnas.2201073119.
Donner, A. J., Ebmeier, C. C., Taatjes, D. J., and Espinosa, J. M. (2010). CDK8 is a positive regulator of transcriptional elongation within the serum response network. Nat. Struct. Mol. Biol 17, 194-201.
Elkabets, M., Vora, S., Juric, D., Morse, N., Mino-Kenudson, M., Muranen, T., Tao, J., Campos, A. B., Rodon, J., Ibrahim, Y. H., et al. (2013). mTORC1 inhibition is required for sensitivity to PI3K p110a inhibitors in PIK3CA-mutant breast cancer. Science translational medicine 5, 196ra199. 10.1126/scitranslmed.3005747.
Fant, C. B., and Taatjes, D. J. (2019). Regulatory functions of the Mediator kinases CDK8 and CDK19. Transcription 10, 76-90. 10.1080/21541264.2018.1556915.
Firestein, R., Bass, A. J., Kim, S. Y., Dunn, I. F., Silver, S. J., Guney, I., Freed, E., Ligon, A. H., Vena, N., Ogino, S., et al. (2008). CDK8 is a colorectal cancer oncogene that regulates beta-catenin activity. Nature 455, 547-551.
Formisano, L., Napolitano, F., Rosa, R., D'Amato, V., Servetto, A., Marciano, R., De Placido, P., Bianco, C., and Bianco, R. (2020). Mechanisms of resistance to mTOR inhibitors. Crit Rev Oncol Hematol 147, 102886. 10.1016/j.critrevonc.2020.102886.
Fryer, C. J., White, J. B., and Jones, K. A. (2004). Mastermind recruits CycC: CDK8 to phosphorylate the Notch ICD and coordinate activation with turnover. Mol. Cell 16, 509-520. S1097276504006409 [pii]; 10.1016/j.molcel.2004.10.014 [doi].
Fukasawa, K., Kadota, T., Horie, T., Tokumura, K., Terada, R., Kitaguchi, Y., Park, G., Ochiai, S., Iwahashi, S., Okayama, Y., et al. (2021). CDK8 maintains stemness and tumorigenicity of glioma stem cells by regulating the c-MYC pathway. Oncogene 40, 2803-2815. 10.1038/s41388-021-01745-1.
Galbraith, M. D., Allen, M. A., Bensard, C. L., Wang, X., Schwinn, M. K., Qin, B., Long, H. W., Daniels, D. L., Hahn, W. C., Dowell, R. D., and Espinosa, J. M. (2013). HIFIA employs CDK8-mediator to stimulate RNAPII elongation in response to hypoxia. Cell 153, 1327-1339. S0092-8674 (13) 00524-2 [pii]; 10.1016/j.cell.2013.04.048 [doi].
Grandjean, J. M., Jiu, A. Y., West, J. W., Aoyagi, A., Droege, D. G., Elepano, M., Hirasawa, M., Hirouchi, M., Murakami, R., Lee, J., et al. (2020). Discovery of 4-Piperazine Isoquinoline Derivatives as Potent and Brain-Permeable Tau Prion Inhibitors with CDK8 Activity. ACS Med Chem Lett 11, 127-132. 10.1021/acsmedchemlett.9b00480.
Hofmann, M. H., Mani, R., Engelhardt, H., Impagnatiello, M. A., Carotta, S., Kerenyi, M., Lorenzo-Herrero, S., Böttcher, J., Scharn, D., Arnhof, H., et al. (2020). Selective and Potent CDK8/19 Inhibitors Enhance NK-Cell Activity and Promote Tumor Surveillance. Mol Cancer Ther 19, 1018-1030. 10.1158/1535-7163.Mct-19-0789.
John Mary, D. J. S., Sikarwar, G., Kumar, A., and Limaye, A. M. (2020). Interplay of ERα binding and DNA methylation in the intron-2 determines the expression and estrogen regulation of cystatin A in breast cancer cells. Mol Cell Endocrinol 504, 110701. 10.1016/j.mce.2020.110701.
Kaur, K., Anant, A., and Asati, V. (2021). Structural Aspects of mTOR Inhibitors: In Progress to Search Potential Compounds. Anticancer Agents Med Chem. 10.2174/1871520621666210720121403.
Khan, M. H., Allerton, R., and Pettit, L. (2015). Hormone Therapy for Breast Cancer in Men. Clin Breast Cancer 15, 245-250. 10.1016/j.clbc.2015.01.007.
Legrier, M. E., Bièche, I., Gaston, J., Beurdeley, A., Yvonnet, V., Déas, O., Thuleau, A., Château-Joubert, S., Servely, J. L., Vacher, S., et al. (2016). Activation of IFN/STAT1 signalling predicts response to chemotherapy in oestrogen receptor-negative breast cancer. Br J Cancer 114, 177-187. 10.1038/bjc.2015.398.
Li, Q., Feng, K., Liu, J., and Ren, Y. (2020). Molecular modeling studies of novel naphthyridine and isoquinoline derivatives as CDK8 inhibitors. J Biomol Struct Dyn, 1-15. 10.1080/07391102.2020.1797537.
Li, Q. L., Gu, F. M., Wang, Z., Jiang, J. H., Yao, L. Q., Tan, C. J., Huang, X. Y., Ke, A. W., Dai, Z., Fan, J., and Zhou, J. (2012). Activation of PI3K/AKT and MAPK pathway through a PDGFRβ-dependent feedback loop is involved in rapamycin resistance in hepatocellular carcinoma. PLOS One 7, e33379. 10.1371/journal.pone.0033379.
Liu, G., Zhang, Z., Song, Q., Guo, Y., Bao, P., and Shui, H. (2020). Circ_0006528 Contributes to Paclitaxel Resistance of Breast Cancer Cells by Regulating miR-1299/CDK8 Axis. Onco Targets Ther 13, 9497-9511. 10.2147/ott.S252886.
Liu, Q., Song, X., Liu, Z., and Yu, Z. (2021). Investigation of Candidate Genes and Pathways in Basal/TNBC Patients by Integrated Analysis. Technol Cancer Res Treat 20, 15330338211019506. 10.1177/15330338211019506.
Ma, D., Chen, X., Shen, X. B., Sheng, L. Q., and Liu, X. H. (2020). Binding patterns and structure-activity relationship of CDK8 inhibitors. Bioorg Chem 96, 103624. 10.1016/j.bioorg.2020.103624.
Mao, B., Zhang, Q., Ma, L., Zhao, D. S., Zhao, P., and Yan, P. (2022). Overview of Research into mTOR Inhibitors. Molecules 27. 10.3390/molecules27165295.
Martínez-González, S., García, A. B., Albarrán, M. I., Cebriá, A., Amezquita-Alves, A., García-Campos, F. J., Martinez-Gago, J., Martínez-Torrecuadrada, J., Muñoz, I., Blanco-Aparicio, C., and Pastor, J. (2020). Pyrido[2,3-b][1,5]benzoxazepin-5 (6H)-one derivatives as CDK8 inhibitors. Eur J Med Chem 201, 112443. 10.1016/j.ejmech.2020.112443.
Martorana, F., Motta, G., Pavone, G., Motta, L., Stella, S., Vitale, S. R., Manzella, L., and Vigneri, P. (2021). AKT Inhibitors: New Weapons in the Fight Against Breast Cancer? Front Pharmacol 12, 662232. 10.3389/fphar.2021.662232.
McDermott, M. S., Chumanevich, A. A., Lim, C. U., Liang, J., Chen, M., Altilia, S., Oliver, D., Rae, J. M., Shtutman, M., Kiaris, H., et al. (2017). Inhibition of CDK8 mediator kinase suppresses estrogen dependent transcription and the growth of estrogen receptor positive breast cancer. Oncotarget 8, 12558-12575. 10.18632/oncotarget. 14894.
Menzl, I., Witalisz-Siepracka, A., and Sexl, V. (2019). CDK8-Novel Therapeutic Opportunities. Pharmaceuticals (Basel) 12. 10.3390/ph12020092.
Meric-Bernstam, F., Akcakanat, A., Chen, H., Do, K. A., Sangai, T., Adkins, F., Gonzalez-Angulo, A. M., Rashid, A., Crosby, K., Dong, M., et al. (2012). PIK3CA/PTEN mutations and Akt activation as markers of sensitivity to allosteric mTOR inhibitors. Clin Cancer Res 18, 1777-1789. 10.1158/1078-0432.Ccr-11-2123.
Millis, S. Z., Ikeda, S., Reddy, S., Gatalica, Z., and Kurzrock, R. (2016). Landscape of Phosphatidylinositol-3-Kinase Pathway Alterations Across 19 784 Diverse Solid Tumors. JAMA Oncol 2, 1565-1573. 10.1001/jamaoncol.2016.0891.
Mishra, R., Patel, H., Alanazi, S., Kilroy, M. K., and Garrett, J. T. (2021). PI3K Inhibitors in Cancer: Clinical Implications and Adverse Effects. Int J Mol Sci 22. 10.3390/ijms22073464.
Pelish, H. E., Liau, B. B., Nitulescu, I. I., Tangpeerachaikul, A., Poss, Z. C., Da Silva, D. H., Caruso, B. T., Arefolov, A., Fadeyi, O., Christie, A. L., et al. (2015). Mediator kinase inhibition further activates super-enhancer-associated genes in AML. Nature 526, 273-276. nature14904 [pii]; 10.1038/nature14904 [doi].
Philip, S., Kumarasiri, M., Teo, T., Yu, M., and Wang, S. (2018). Cyclin-Dependent Kinase 8: A New Hope in Targeted Cancer Therapy? J Med Chem. 10.1021/acs.jmedchem. 7b00901.
Rascón-Cruz, Q., Espinoza-Sánchez, E. A., Siqueiros-Cendón, T. S., Nakamura-Bencomo, S. I., Arévalo-Gallegos, S., and Iglesias-Figueroa, B. F. (2021). Lactoferrin: A Glycoprotein Involved in Immunomodulation, Anticancer, and Antimicrobial Processes. Molecules 26. 10.3390/molecules26010205.
Rzymski, T., Mikula, M., Zylkiewicz, E., Dreas, A., Wiklik, K., Golas, A., Wojcik, K., Masiejczyk, M., Wrobel, A., Dolata, I., et al. (2017). SEL 120-34A is a novel CDK8 inhibitor active in AML cells with high levels of serine phosphorylation of STAT1 and STAT5 transactivation domains. Oncotarget 8, 33779-33795. 10.18632/oncotarget. 16810.
Schneider, L., Liu, J., Zhang, C., Azoitei, A., Meessen, S., Zheng, X., Cremer, C., Gorzelanny, C., Kempe-Gonzales, S., Brunner, C., et al. (2021). The Role of Interleukin-1-Receptor-Antagonist in Bladder Cancer Cell Migration and Invasion. Int J Mol Sci 22. 10.3390/ijms22115875.
Serrao, A., Jenkins, L. M., Chumanevich, A. A., Horst, B., Liang, J., Gatza, M. L., Lee, N. Y., Roninson, I. B., Broude, E. V., and Mythreye, K. (2018). Mediator kinase CDK8/CDK19 drives YAP1-dependent BMP4-induced EMT in cancer. Oncogene 37, 4792-4808. 10.1038/s41388-018-0316-y.
Sharko, A. C., Lim, C. U., McDermott, M. S. J., Hennes, C., Philavong, K. P., Aiken, T., Tatarskiy, V. V., Roninson, I. B., and Broude, E. V. (2021). The Inhibition of CDK8/19 Mediator Kinases Prevents the Development of Resistance to EGFR-Targeting Drugs. Cells 10. 10.3390/cells10010144.
Steinparzer, I., Sedlyarov, V., Rubin, J. D., Eislmayr, K., Galbraith, M. D., Levandowski, C. B., Vcelkova, T., Sneezum, L., Wascher, F., Amman, F., et al. (2019). Transcriptional Responses to IFN-γ Require Mediator Kinase-Dependent Pause Release and Mechanistically Distinct CDK8 and CDK19 Functions. Mol Cell 76, 485-499.e488. 10.1016/j.molcel.2019.07.034.
Tsutsui, T., Fukasawa, R., Tanaka, A., Hirose, Y., and Ohkuma, Y. (2011). Identification of target genes for the CDK subunits of the Mediator complex. Genes Cells 16, 1208-1218. 10.1111/j.1365-2443.2011.01565.x [doi].
Vitale, S. R., Martorana, F., Stella, S., Motta, G., Inzerilli, N., Massimino, M., Tirrò, E., Manzella, L., and Vigneri, P. (2021). PI3K inhibition in breast cancer: Identifying and overcoming different flavors of resistance. Crit Rev Oncol Hematol 162, 103334. 10.1016/j.critrevonc.2021.103334.
Wellberg, E. A., Rudolph, M. C., Lewis, A. S., Padilla-Just, N., Jedlicka, P., and Anderson, S. M. (2014). Modulation of tumor fatty acids, through overexpression or loss of thyroid hormone responsive protein spot 14 is associated with altered growth and metastasis. Breast Cancer Res 16, 481. 10.1186/s13058-014-0481-z.
Wells, W. A., Schwartz, G. N., Morganelli, P. M., Cole, B. F., Gibson, J. J., and Kinlaw, W. B. (2006). Expression of “Spot 14” (THRSP) predicts disease free survival in invasive breast cancer: immunohistochemical analysis of a new molecular marker. Breast Cancer Res Treat 98, 231-240. 10.1007/s10549-005-9154-z.
Woo, S. U., Sangai, T., Akcakanat, A., Chen, H., Wei, C., and Meric-Bernstam, F. (2017). Vertical inhibition of the PI3K/Akt/mTOR pathway is synergistic in breast cancer. Oncogenesis 6, e385. 10.1038/oncsis.2017.86.
Wu, D., Zhang, Z., Chen, X., Yan, Y., and Liu, X. (2021). Angel or Devil?-CDK8 as the new drug target. Eur J Med Chem 213, 113043. 10.1016/j.ejmech.2020.113043.
Xi, M., Chen, T., Wu, C., Gao, X., Wu, Y., Luo, X., Du, K., Yu, L., Cai, T., Shen, R., and Sun, H. (2019). CDK8 as a therapeutic target for cancers and recent developments in discovery of CDK8 inhibitors. Eur J Med Chem 164, 77-91. 10.1016/j.ejmech.2018.11.076.
Yu, M., Teo, T., Yang, Y., Li, M., Long, Y., Philip, S., Noll, B., Heinemann, G. K., Diab, S., Eldi, P., et al. (2021). Potent and orally bioavailable CDK8 inhibitors: Design, synthesis, structure-activity relationship analysis and biological evaluation. Eur J Med Chem 214, 113248. 10.1016/j.ejmech.2021.113248.
Zhou H Y, Huang S L. Current development of the second generation of mTOR inhibitors as anticancer agents. Chin J Cancer. 2012 January; 31 (1): 8-18. doi: 10.5732/cjc.011.10281. Epub 2011 Nov. 4. PMID: 22059905; PMCID: PMC3249493.

1. A method for treating a subject, the method comprising administering to the subject a CDK8/19 inhibitor and a mTOR inhibitor, wherein the subject is in need of a treatment for a cancer.

2. The method of claim 1, wherein the subject has a solid tumor.

3. The method of claim 2, wherein the solid tumor has a genetic alteration associated with PI3K/AKT/mTOR dysregulation.

4. The method of claim 1, wherein the cancer is a breast cancer.

5. The method of claim 4, wherein the cancer is a triple-negative breast cancer.

6. The method of claim 4, wherein the breast cancer is estrogen receptor positive.

7. The method of claim 4, wherein the breast cancer is HER2-positive.

8. The method of claim 1, wherein the CDK8/19 inhibitor comprises a member selected from the group consisting of SNX631, SNX631-6, Senexin B, Senexin C, BI-1347, cortistatin A, SEL120-34A, and any combination thereof.

9. The method of claim 1, wherein the mTOR inhibitor comprises a member selected from the group consisting of everolimus, temsirolimus, sirolimus, ridaforolimus, umirolimus, gedatolisib, and any combination thereof.

10. (canceled)

11. The method of claim 1, wherein the Combination Index (CI) for the CDK8/19 inhibitor and the mTOR inhibitor in a tumor cell line at an effective dose is less than 1 as determined by measuring the relative cell number by sulforhodamine B (SRB) assay at 570 nm wavelength light absorbance.

12. The method of claim 11, wherein the mTOR inhibitor kills, inhibits growth, or inhibits proliferation of cells of the tumor cell line.

13. (canceled)

14. The method of claim 1, wherein the CDK8/19 inhibitor is administered with the mTOR inhibitor by a pharmaceutical composition comprising the CDK8/19 inhibitor and the mTOR inhibitor.

15. The method of claim 1, wherein the CDK8/19 inhibitor is administered with the mTOR inhibitor by a kit comprising the CDK8/19 inhibitor and the mTOR inhibitor.

16. The method of claim 1, wherein the administration of the CDK8/19 inhibitor prevents resistance to the mTOR inhibitor.

17. The method of claim 16, wherein the subject has a solid tumor.

18. The method of claim 17, wherein the solid tumor has a genetic alteration associated with PI3K/AKT/mTOR dysregulation.

19.-23. (canceled)

24. The method of claim 16, wherein the mTOR inhibitor comprises a member selected from the group consisting of everolimus, temsirolimus, sirolimus, ridaforolimus, umirolimus, gedatolisib, and any combination thereof.

25. The method of claim 16, wherein the CDK8/19 inhibitor comprises a member selected from the group consisting of SNX631, SNX631-6, Senexin B, Senexin C, BI-1347, cortistatin A, SEL120-34A, and any combination thereof and the mTOR inhibitor comprises a member selected from the group consisting of everolimus, temsirolimus, sirolimus, ridaforolimus, umirolimus, gedatolisib, and any combination thereof.

26.-30. (canceled)

31. A pharmaceutical composition comprising a CDK8/19 inhibitor, a mTOR inhibitor, and a pharmaceutically acceptable carrier, excipient, or diluent.

32.-51. (canceled)

52. A kit comprising a container that contains a CDK8/19 inhibitor and a mTOR inhibitor, wherein the contents of the kit are for administration to a subject in need of a treatment for a cancer.

51.-69. (canceled)
